SEQUENCE LISTING (2 sequences)

SEQ ID NO: 1 - protein, 12 aa, Artificial Sequence
  synthetic
CAVRPARQNFVF

SEQ ID NO: 2 - protein, 12 aa, Artificial Sequence
  synthetic
CASSETGWGAFF

(19) United States
(12) Patent Application Publication
(10) Pub. No.: US 2023/0036952 A1
(43) Pub. Date: Feb. 2, 2023

(54) METHODS OF PRODUCING T CELL POPULATIONS USING INDUCED PLURIPOTENT STEM CELLS

(71) Applicant: The United States of America,as represented by the Secretary,Department of Health and Human Services, Bethesda, MD (US)

(72) Inventors: Meghan L. Good, Derwood, MD (US); Rafiqul SM Islam, Bethesda, MD (US); Naritaka Tamaoki, North Bethesda, MD (US); Raul E. Vizcardo, North Bethesda, MD (US); Nicholas P. Restifo, Chevy Chase, MD (US)

(73) Assignee: The United States of America,as represented by the Secretary,Department of Health and Human Services, Bethesda, MD (US)

(21) Appl. No.: 17/790,939

(22) PCT Filed: Jan. 7, 2021
(86) PCT No.: PCT/US2021/012444

§ 371 Date: Jul. 5, 2022

Related U.S. Application Data
(60) Provisional application No. 62/957,939, filed on Jan. 7, 2020.

(51) Int. Cl. C12N 5/0783; A61P 35/00; A61P 37/02

(52) U.S. Cl. CPC C12N 5/0636; A61P 35/00; A61P 37/02; C12N 2501/2302; C12N 2501/2307; C12N 2501/2312; C12N 2501/2315; C12N 2506/45

(57) ABSTRACT

Provided are methods of producing an isolated population of T cells for adoptive cell therapy. Also provided are related isolated populations of cells, pharmaceutical compositions, and methods of treating or preventing cancer, infections, and autoimmune conditions in a patient.

### CROSS-REFERENCE TO RELATED APPLICATION

This patent application claims the benefit of co-pending U.S. Provisional Patent Application No. 62/957,939 filed Jan. 7, 2020, which is incorporated by reference in its entirety herein.

### STATEMENT REGARDING FEDERALLY SPONSORED RESEARCH OR DEVELOPMENT

This invention was made with Government support under project number ZO1ZIA BC010763 by the National Institutes of Health, National Cancer Institute. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 784 Byte ASCII (Text) file named “750925_ST25.txt,” dated Dec. 29, 2020.

### BACKGROUND OF THE INVENTION

Adoptive cell therapy (ACT) using cancer-reactive T cells can produce positive clinical responses in some cancer patients. Nevertheless, several obstacles to the successful use of ACT for the treatment of cancer and other conditions remain. For example, the current methods used to produce cancer-reactive T cells require significant time and may not readily identify the desired T cell receptors (TCRs) that bind cancer targets. Accordingly, there is a need for methods of obtaining an improved isolated population of cells for ACT.

### BRIEF SUMMARY OF THE INVENTION

An embodiment of the invention provides a method of producing an isolated population of T cells for ACT, the method comprising a) providing a patient sample, wherein the patient sample is (1) a sample containing T cells from a patient having a tumor, (2) a sample containing T cells from a patient with an infection, or (3) a sample containing T cells from a patient with an autoimmune condition, wherein the T cells comprise at least one TCR, wherein the TCR comprises an alpha-chain and beta-chain pair; b) separating T cells from other cells of the patient sample of a) to produce separated T cells; c) culturing the separated T cells of b) to produce T cell induced pluripotent stem cells (iPSCs); d) culturing the T cell iPSCs of c) to produce CD4⁻CD8⁻(double negative) T cells; e) screening the CD4⁻CD8⁻(double negative) T cells to identify one or more CD4⁻CD8⁻(double negative) T cells that comprise a TCR alpha-chain and beta-chain pair having antigenic specificity for an antigen of interest, wherein the antigen of interest is (1) a cancer antigen, (2) a pathogen antigen, or (3) an antigen causing autoimmunity; and f) differentiating the double negative T cell(s) identified in e) as comprising the TCR alpha-chain and beta-chain pair having antigenic specificity for the antigen of interest into naïve T cells and expanding the number of naïve T cells to produce an isolated population of T cells for adoptive cell therapy.

Further embodiments of the invention provide isolated populations of T cells produced by the method, related pharmaceutical compositions, and related methods of treating or preventing cancer, infections, and autoimmune conditions in a patient.

### BRIEF DESCRIPTION OF THE SEVERAL VIEWS OF THE DRAWING(S)

FIG. 1 is a schematic representation of an embodiment of the inventive methods.

FIG. 2 is a graph showing the percentage of productive frequency for CD8 alone or tumor infiltrating lymphocyte (TIL)-induced pluripotent stem cell (iPSC) lines for TCR AV21-01, CAVRPARQNFVF (SEQ ID NO: 1). 23 iPSC lines from TIL, specific for mutated GBAS peptide, were produced. All of the TIL-iPSC lines generated showed only one inherited rearrangement of a TCR alpha-chain and beta-chain pair, which was identical to that of the cell source.

FIG. 3 is a graph showing the percentage of productive frequency for CD8 alone or TIL-iPSC lines for TCR BV02-01*01, CASSETGWGAFF (SEQ ID NO: 2). 23 iPSC lines from TIL, specific for mutated GBAS peptide, were produced. All of the TIL-iPSC lines generated showed only one inherited rearrangement of TCR alpha-chain and TCR beta-chain, which was identical to that of the cell source.

FIG. 4 shows representative fluorescence-activated cell sorting (FACS) data showing that 5 of the 23 TIL-iPSC lines differentiated into T lineage cells in vitro. Gating was as indicated in the figure. All 5 of the selected TIL-iPSC lines produced CD4+CD8+ double positive T cells at day 35.

FIG. 5 is a line graph showing the percentage of CD3+TCRab+open-repertoire iPSC (OR-iPSC) per day.

FIG. 6 is a line graph showing the percentage of CD3+TCRab+TIL-iPSC per day.

FIG. 7 shows representative FACS data showing precocious expression of TCR complex in human TIL-iPSC derived immature T cells (TIL-iPSC line #15). Gating was as indicated in the figure.

FIGS. 8A and 8B are graphs showing that TIL-iPSC derived CD4+CD8+(double positive) T cells do not show specificity against tumor mutations. The double positive T cells were activated (4-1BB+) by phorbol 12-myristate 13-acetate (PMA) stimulation but not by mutated peptide as compared to the TIL cell source. “WT” refers to wild type and “Mut” to mutated peptide. FIG. 8A is CD8 alone and FIG. 8B is TIL-iPSC derived double positive cells.

FIGS. 9A and 9B are graphs showing that TIL-iPSC derived double negative T cells have high tumor-antigen specific reactivity. The double negative T cells were activated (4-1BB+) by PMA stimulation and responded to mutated peptide but not to wild type peptide as the parental TILs did. “WT” refers to wild type and “Mut” to mutated peptide. FIG. 9A is CD8 alone and FIG. 9B is TIL-iPSC derived double negative cells.

FIGS. 10A-10C are graphs showing that DN cells produce interferon gamma (IFNγ) in response to mutant peptide but not wild type peptide (FIG. 10B), similar to CD8 TIL clone (FIG. 10A). DP cells show nonspecific production of INFγ across all conditions (FIG. 10C).

### DETAILED DESCRIPTION OF THE INVENTION

New methods have been discovered that produce a high number of T cells that a) contain a TCR alpha-chain and beta-chain pair that are present in T cells that are separated from tumor cells of a tumor sample and b) have desirable specificity for tumor antigens in the tumor cells of the tumor sample. These methods may provide any one or more of a variety of advantages. These advantages may include, for example, not requiring prediction algorithms and secondary screenings to determine the appropriate alpha-chain and beta-chain pair that was present in the T cells that were separated from the tumor cells of the tumor sample. Further, the methods do not require extraction of the DNA and RNA from the tumor cells of the tumor sample or sequencing of the extracted sequences. The methods save significant amounts of time and may provide significant cost savings allowing ACT to be available to more patients. In addition, the methods can be used to identify viral or bacterial antigen specific TCR alpha-chain and beta-chain pairs to treat infections (e.g., HIV) and to identify endogenous TCR alpha-chain and beta-chain pairs causing autoimmunity in patients.

iPSC technology allows the reprogramming of somatic cells into an embryonic stem cell-like stage, which can be expanded indefinitely and retain the potential to differentiate into any type of somatic cell. Reprogramming of TILs can be beneficial because the TCRs providing the tumor specificity of TILs are produced by genomic recombination and inherited in the generated iPSC. Further differentiation of human TIL-iPSC into double negative immature T cells leads to precocious expression of the TCR complex, which can recognize target peptide. TIL-iPSC derived double negative T cells show a higher affinity to target peptides than other iPSC-derived T cells previously generated (double positive or CD8 single positive T cells). Therefore, the generation of TIL-iPSC and their further validation in double negative T cell stage can serve to develop a new screening method to find tumor-antigen specific TCRs from bulk populations of TILs. These TCRs can be subsequently cloned and used in ACT treatments.

Conventional methods suffer from several disadvantages, including limited efficiency of sequencing due to the small number of reactive T cells. This limitation leads to the necessity to use algorithms to determine the possible combinations of TCR-alpha and TCR-beta genes from a bulk population. The conventional methods also require cloning of the potential TCR combinations and subsequent validation of TCRs by screening.

An embodiment of the invention provides a method of producing an isolated population of T cells. The method may comprise providing a patient sample containing T cells and cells from a patient having a tumor, infection, or autoimmune condition, e.g., (1) a sample containing T cells from a patient having a tumor, (2) a sample containing T cells from a patient with an infection, or (3) a sample containing T cells from a patient with an autoimmune condition. In an embodiment of the invention, the sample containing T cells from a patient having a tumor is a tumor sample from the patient. The tumor sample can be any suitable tumor sample (liquid or solid) that has T cells present in a sufficient quantity to produce at least one TCR for sequencing. The tumor sample may be obtained by, for example, resection, blood draw, leukapheresis, or another suitable technique.

The T cells comprise at least one TCR. The T cells can comprise about 1, about 2, about 3, about 4, about 5, about 6, about 7, about 8, about 9, or about 10 TCRs, or any range of numbers of the foregoing (e.g., about 1 to about 10, about 2 to about 10, about 1 to about 9, about 2 to about 9, etc.).

A TCR generally comprises two polypeptides (i.e., polypeptide chains), such as an alpha-chain of a TCR, a beta-chain of a TCR, a γ-chain of a TCR, a δ-chain of a TCR, or a combination thereof. Such polypeptide chains of TCRs are known in the art. The antigen-specific TCR can comprise any amino acid sequences, provided that the TCR can specifically bind to and immunologically recognize an antigen of interest, such as (1) a cancer antigen, (2) a pathogen antigen, (3) an antigen causing autoimmunity, or an epitope thereof.

The method may further comprise separating the T cells from the other cells of the patient sample to produce a separated population of T cells and a separated population of patient sample cells that are not T cells. This separation step may be accomplished using any suitable technique, for example, FACS, magnetic separation (MACs), acoustic separation, and electrokinetic separation.

The population of T cells separated from the other patient sample cells may include any type of T cells. The T cell may be a human T cell. The T cell can be any type of T cell and can be of any developmental stage, including but not limited to, CD4⁺/CD8⁺ double positive T cells, CD4⁺ T cells, e.g., Th₁ and Th₂ cells, CD8⁺ T cells (e.g., cytotoxic T cells), Th₉ cells, TIL, memory T cells, naïve T cells, and the like. The T cell may be a CD8⁺ T cell or a CD4⁺ T cell. Naïve T cells are mature T cells that have not encountered a cognate antigen within their periphery. Naïve T cells are commonly characterized by the surface expression of L-selectin (CD62L) and C-C Chemokine receptor type 7 (CCR7), the absence of the activation markers CD25, CD44 or CD69, the absence of memory CD45RO isoform, and/or expression of functional IL-7 receptors, including subunits IL-7 receptor-α, CD127, and common-γ chain, CD132. In a preferred embodiment, the T cells are tumor infiltrating lymphocytes (TIL).

The method may further comprise separating T cells from other cells of the patient sample to produce separated T cells. The T cells can be separated from other cells of the patient sample using any suitable technique, for example, FACS, magnetic separation (MACs), acoustic separation, and electrokinetic separation.

The method may further comprise culturing the separated T cells to produce T cell iPSCs. The T cells can be cultured to produce T cell iPSCs using any suitable technique, for example, the separated T cells can receive stimulation from an anti-CD3 and/or an anti-CD28 antibodie(s) and/or be transduced (e.g., with a vector) with sequences of the Yamanaka factors (i.e., Kruppel-like factor 4 (Klf4), Sry-related HMG-box gene 2 (Sox2), Octamer-binding transcription factor 3/4 (Oct3/4), and MYC protooncogene (c-Myc)) and SV40 (see, e.g., Vizcardo, et al., Cell Stem Cell, 12: 31-36 (2013)). The T cells can be cultured in the presence of interleukin-2 (IL-2), interleukin-7 (IL-7), interleukin-15 (IL-15), interleukin-12 (IL-12), or a combination of two or more of the foregoing. Alternatively, iPSC can be generated by RNA expression, protein delivery, chemical induction of reprogramming genes, activation by upstream or downstream targeting of gene pathways essential for reprogramming (e.g. Sox2, KLf4, Oct4, Nanog, etc), or any combination of these methods.

The method may further comprise culturing the T cell iPSCs to produce CD4⁻CD8⁻(double negative) T cells. Double negative T cells do not express the CD4 or CD8 co-receptor. Double negative T cells are differentiated from common lymphoid progenitor (CLP) cells and engraft in the thymus. The T cell iPSCs can be cultured to produce CD4-CD8− (double negative) T cells using any suitable technique.

The method may further comprise screening the CD4⁻CD8⁻(double negative) T cells to identify one or more CD4⁻CD8⁻(double negative) T cells that comprise a TCR alpha-chain and beta-chain pair having antigenic specificity for an antigen of interest. The cells can be screened using any suitable technique.

The method may further comprise differentiating the double negative T cells identified as comprising the TCR alpha-chain and beta-chain pair having antigenic specificity for an antigen of interest into naïve T cells. The cells can be differentiated using any suitable technique.

In an embodiment of the invention, the method comprises obtaining sequence(s) that encodes the alpha-chain and beta-chain pair of the TCR. For this step, nested PCR or alignment by adaptive screening may be used, or another suitable technique.

In an embodiment of the invention, the method comprises transducing naïve PMBCs with the sequence of the alpha-chain and beta-chain pair of the TCR, for example, by using vector(s). For example, retroviral vector(s) as described in Johnson et al., Blood, 114: 535-546 (2009) can be used. Alternatively, the following techniques may be used: (1) using targeted integration as described in, for example, Roth et al., Nature, 559: 405-409 (2018); (2) using a transposon as described in, for example, Peng et al., Gene Ther., 16: 1042-1049 (2009); and (3) using transiently expressed RNA (e.g., mRNA) as described in, for example, Zhao et al., Mol. Ther., 13: 151-159 (2006), or another suitable technique. While the PBMCs may be allogeneic, in a preferred embodiment, the PBMCs are autologous to the patient.

The PBMC used to provide an isolated population of cells for ACT can be any suitable PBMC, for example, peripheral blood lymphocytes (PBLs), B cells, dendritic cells, or a combination of two or more of the foregoing. In a preferred embodiment, the PBMC is a T cell.

In an embodiment of the invention, the method allows for a patient to receive a population of cells for ACT (with one or more TCRs specific for the antigen of interest) only about 30 or fewer days after a patient sample (e.g., tumor sample) is removed from the patient. For example, the patient may receive a population of cells for ACT (with one or more TCRs specific for the antigen of interest) only about 28 or fewer, about 26 or fewer, about 24 or fewer, about 22 or fewer, about 20 or fewer, about 18 or fewer, about 16 or fewer, about 15 or fewer, about 14 or fewer, about 13 or fewer, about 12 or fewer, about 11 or fewer, about 10 or fewer, about 9 or fewer, about 8 or fewer, about 7 or fewer, about 6 or fewer, about 5 or fewer, about 4 or fewer, about 3 or fewer, or about 2 or fewer days after a sample is removed from the patient.

In an embodiment of the invention, the TCRs of the T cells identified by the inventive methods have antigenic specificity for a tumor (i.e., cancer antigen) of the tumor cells. In a further embodiment of the invention, the TCRs of the T cells identified by the inventive methods specifically bind to the one or more tumor antigens of the tumor cells. The terms “cancer antigen” and “tumor antigen,” as used herein, refers to any molecule (e.g., protein, polypeptide, peptide, lipid, carbohydrate, etc.) solely or predominantly expressed or over-expressed by a tumor cell or cancer cell, such that the antigen is associated with the tumor or cancer. The cancer antigen can additionally be expressed by normal, non-tumor, or non-cancerous cells. However, in such cases, the expression of the cancer antigen by normal, non-tumor, or non-cancerous cells is not as robust as the expression by tumor or cancer cells. In this regard, the tumor or cancer cells can over-express the antigen or express the antigen at a significantly higher level, as compared to the expression of the antigen by normal, non-tumor, or non-cancerous cells. Also, the cancer antigen can additionally be expressed by cells of a different state of development or maturation. For instance, the cancer antigen can be additionally expressed by cells of the embryonic or fetal stage, which cells are not normally found in an adult host. Alternatively, the cancer antigen can be additionally expressed by stem cells or precursor cells, which cells are not normally found in an adult host. In an embodiment, the TCR alpha-chain and beta-chain pair has specificity for a melanoma antigen.

The cancer antigen can be an antigen expressed by any cell of any cancer or tumor, including the cancers and tumors described herein. The cancer antigen may be a cancer antigen of only one type of cancer or tumor, such that the cancer antigen is associated with or characteristic of only one type of cancer or tumor. Alternatively, the cancer antigen may be a cancer antigen (e.g., may be characteristic) of more than one type of cancer or tumor. For example, the cancer antigen may be expressed by both breast and prostate cancer cells and not expressed at all by normal, non-tumor, or non-cancer cells. Cancer antigens are known in the art and include, for instance, CXorf61, mesothelin, CD19, CD22, CD276 (B7H3), gp100, MART-1, Epidermal Growth Factor Receptor Variant III (EGFRVIII), TRP-1, TRP-2, tyrosinase, NY-ESO-1 (also known as CAG-3), MAGE-1, MAGE-3, etc.

The cancer may be any cancer, including any of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, cholangiocarcinoma, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In certain preferred embodiments, the antigen-specific receptor has specificity for a melanoma antigen.

In an embodiment of the invention, the cancer antigen is a cancer neoantigen. A cancer neoantigen is an immunogenic mutated amino acid sequence which is encoded by a cancer-specific mutation. Cancer neoantigens are not expressed by normal, non-cancerous cells and may be unique to the patient. ACT with T cells which have antigenic specificity for a cancer neoantigen may provide a “personalized” therapy for the patient.

In some embodiments, the tumor sample comes from a patient which has been immunized with an antigen of, or specific for, a cancer. The patient may be immunized prior to obtaining the tumor sample from the patient. In this way, the tumor can include T cells induced to have specificity for the cancer to be treated, or can include a higher proportion of cells specific for the cancer.

In an embodiment of the invention, the TCRs of the T cells identified by the inventive methods have antigenic specificity for an antigen present on an infection-causing microorganism or virus (e.g., a virus, bacteria, fungus, or parasite). For example, the TCRs of the T cells identified by the inventive methods can have antigenic specificity for an antigen present on a virus, e.g., HIV.

In a further embodiment of the invention, the TCRs of the T cells identified by the inventive methods have specificity for endogenous TCR alpha-chain and beta-chain pairs that cause autoimmunity in patients.

The terms “nucleic acid” and “polynucleotide,” as used herein, refer to a polymeric form of nucleotides of any length, either ribonucleotides (RNA) or deoxyribonucleotides (DNA). These terms refer to the primary structure of the molecule, and thus include double- and single-stranded DNA, double- and single-stranded RNA, and double-stranded DNA-RNA hybrids. The terms include, as equivalents, analogs of either RNA or DNA made from nucleotide analogs and modified polynucleotides such as, though not limited to, methylated and/or capped polynucleotides. In an embodiment of the invention, the nucleic acid is complementary DNA (cDNA).

The term “nucleotide” as used herein refers to a monomeric subunit of a polynucleotide that consists of a heterocyclic base, a sugar, and one or more phosphate groups. The naturally occurring bases (guanine (G), adenine (A), cytosine (C), thymine (T), and uracil (U)) are typically derivatives of purine or pyrimidine, though the invention includes the use of naturally and non-naturally occurring base analogs. The naturally occurring sugar is the pentose (five-carbon sugar) deoxyribose (which forms DNA) or ribose (which forms RNA), though the invention includes the use of naturally and non-naturally occurring sugar analogs. Nucleic acids are typically linked via phosphate bonds to form nucleic acids or polynucleotides, though many other linkages are known in the art (e.g., phosphorothioates, boranophosphates, and the like). Methods of preparing polynucleotides are within the ordinary skill in the art (Green and Sambrook, Molecular Cloning: A Laboratory Manual, (4th Ed.) Cold Spring Harbor Laboratory Press, New York (2012)).

The nucleic acids described herein can be incorporated into recombinant expression vector(s). For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors may not be naturally-occurring as a whole. However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector. Examples of recombinant expression vectors that may be useful in the inventive methods include, but are not limited to, plasmids, viral vectors (e.g., one or more retroviral vectors, gamma-retroviral vectors, or lentiviral vectors), and transposons. The vector(s) may then, in turn, be introduced into the cells by any suitable technique such as, e.g., gene editing, transfection, transformation, or transduction as described, for example, Green and Sambrook, Molecular Cloning: A Laboratory Manual (4th Ed.), Cold Spring Harbor Laboratory Press (2012). Many transfection techniques are known in the art and include, for example, calcium phosphate DNA co-precipitation; DEAE-dextran; electroporation; cationic liposome-mediated transfection; tungsten particle-facilitated microparticle bombardment; and strontium phosphate DNA co-precipitation. Phage or viral vector(s) can be introduced into host cells, after growth of infectious particles in suitable packaging cells, many of which are commercially available.

In an embodiment of the invention, the method further comprises expanding the number of naïve T cells to produce an isolated population of T cells for ACT. This expansion can be done with one or both of (a) one or more cytokines and (b) one or more non-specific T cell stimuli. Examples of non-specific T cell stimuli include, but are not limited to, one or more of irradiated allogeneic feeder cells, irradiated autologous feeder cells, anti-CD3 antibodies (e.g., OKT3 antibody), anti-4-1BB antibodies, and anti-CD28 antibodies. In preferred embodiment, the non-specific T cell stimulus may be anti-CD3 antibodies and anti-CD28 antibodies conjugated to beads. Any one or more cytokines may be used in the inventive methods. Exemplary cytokines that may be useful for expanding the numbers of cells include interleukin (IL)-2, IL-7, IL-21, IL-15, or a combination thereof.

Expansion of the numbers of cells can be accomplished by any of a number of methods as are known in the art as described in, for example, U.S. Pat. Nos. 8,034,334; 8,383,099; and U.S. Patent Application Publication No. 2012/0244133. For example, expansion of the numbers of cells may be carried out by culturing the cells with OKT3 antibody, IL-2, and feeder PBMC (e.g., irradiated allogeneic PBMC).

An embodiment of the invention further provides an isolated or purified population of T cells produced by any of the inventive methods described herein. The populations of T cells produced by the inventive methods may provide any one or more of many advantages.

The population of cells produced by according to the inventive methods can be a heterogeneous population comprising the cells described herein, in addition to at least one other cell, e.g., a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells produced by the inventive methods can be a substantially homogeneous population, in which the population comprises mainly of the cells, e.g., T cells described herein. The population also can be a clonal population of cells, in which all cells of the population are clones of a single cell, e.g., T cell. In one embodiment of the invention, the population of cells is a clonal population comprising cells, e.g., T cells comprising a recombinant expression vector encoding the antigen-specific receptor as described herein.

The inventive isolated or purified population of cells produced according to the inventive methods may be included in a composition, such as a pharmaceutical composition. In this regard, an embodiment of the invention provides a pharmaceutical composition comprising the isolated or purified population of cells described herein and a pharmaceutically acceptable carrier.

Preferably, the carrier is a pharmaceutically acceptable carrier. With respect to pharmaceutical compositions, the carrier can be any of those conventionally used for the administration of cells. Such pharmaceutically acceptable carriers are well-known to those skilled in the art and are readily available to the public. It is preferred that the pharmaceutically acceptable carrier be one which has no detrimental side effects or toxicity under the conditions of use.

The choice of carrier will be determined in part by the particular method used to administer the population of cells. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the population of cells, and in certain instances, a particular route can provide a more immediate and more effective response than another route.

Preferably, the population of cells is administered by injection, e.g., intravenously. A suitable pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water), NORMOSOL electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

The T cells administered to the patient can be allogeneic or autologous to the patient. In “autologous” administration methods, cells are removed from a patient, stored (and optionally modified), and returned back to the same patient. In “allogeneic” administration methods, a patient receives cells from a genetically similar, but not identical, donor. Preferably, the T cells are autologous to the patient. Autologous cells may, advantageously, reduce or avoid the undesirable immune response that may target an allogeneic cell such as, for example, graft-versus-host disease.

In the instance that the T cell(s) are autologous to the patient, the patient can be immunologically naïve, immunized, diseased, or in another condition prior to isolation of the sample from the patient. In some instances, it is preferable for the method to comprise immunizing the patient with an antigen of interest prior to isolating the sample from the patient.

In accordance with an embodiment of the invention, a patient with cancer can be therapeutically immunized with an antigen from, or associated with, that cancer, including immunization via a vaccine. While not desiring to be bound by any particular theory or mechanism, the vaccine or immunogen is provided to enhance the patient's immune response to the cancer antigen present in the tumor. Such a therapeutic immunization includes, but is not limited to, the use of recombinant or natural cancer proteins, peptides, or analogs thereof, or modified cancer peptides, or analogs thereof that can be used as a vaccine therapeutically as part of adoptive immunotherapy. The vaccine or immunogen, can be a cell, cell lysate (e.g., from cells transfected with a recombinant expression vector), a recombinant expression vector, or antigenic protein or polypeptide. Alternatively, the vaccine, or immunogen, can be a partially or substantially purified recombinant cancer protein, polypeptide, peptide or analog thereof, or modified proteins, polypeptides, peptides or analogs thereof. The protein, polypeptide, or peptide may be conjugated with lipoprotein or administered in liposomal form or with adjuvant. Preferably, the vaccine comprises one or more of (i) the cancer antigen for which the antigen-specific receptor has antigenic specificity, (ii) an epitope of the antigen, and (iii) a vector encoding the antigen or the epitope.

For purposes of the invention, the dose, e.g., number of cells administered should be sufficient to effect, e.g., a therapeutic or prophylactic response, in the patient over a reasonable time frame. For example, the number of cells administered should be sufficient to bind to an antigen of interest or treat or prevent cancer, infections, and/or an autoimmune condition in a period of from about 2 hours or longer, e.g., 12 to 24 or more hours, from the time of administration. In certain embodiments, the time period could be even longer. The number of cells administered will be determined by, e.g., the efficacy of the particular population of cells to be administered and the condition of the animal (e.g., human), as well as the body weight of the animal (e.g., human) to be treated.

Many assays for determining an administered number of cells are known in the art. For purposes of the invention, an assay, which comprises comparing the extent to which target cells are lysed or one or more cytokines such as, e.g., IFN-γ and IL-2 is secreted upon administration of a given number of such cells to a patient among a set of patients of which is each given a different number of the cells, e.g., T cells, could be used to determine a starting number to be administered to a patient. The extent to which target cells are lysed or cytokines such as, e.g., IFN-γ and IL-2 are secreted upon administration of a certain number can be assayed by methods known in the art. Secretion of cytokines such as, e.g., IL-2, may also provide an indication of the quality (e.g., phenotype and/or effectiveness) of a T cell preparation.

The number of cells administered also will be determined by the existence, nature and extent of any adverse side effects that might accompany the administration of a particular population of cells. Typically, the attending physician will decide the number of cells with which to treat each individual patient, taking into consideration a variety of factors, such as age, body weight, general health, diet, sex, route of administration, and the severity of the condition being treated. By way of example and not intending to limit the invention, the number of cells, e.g., T cells, to be administered can be about 10×10⁶ to about 10×10¹¹ cells per infusion, about 10×10⁹ cells to about 10×10¹¹ cells per infusion, or 10×10⁷ to about 10×10⁹ cells per infusion.

It is contemplated that the populations of T cells produced according to the inventive methods can be used in methods of treating or preventing cancer in a patient. In this regard, an embodiment of the invention provides a method of treating or preventing cancer in a patient, comprising (i) administering to the patient the cells produced according to any of the methods described herein or (ii) administering to the patient any of the isolated populations of cells or pharmaceutical compositions described herein; in an amount effective to treat or prevent cancer in the patient.

In an embodiment of the invention, the method of treating or preventing cancer may comprise administering the cells or pharmaceutical composition to the patient in an amount effective to reduce metastases in the patient. For example, the inventive methods may reduce metastatic nodules in the patient.

It is also contemplated that the populations of T cells produced according to the inventive methods can be used in methods of treating or preventing an infection in a patient. In this regard, an embodiment of the invention provides a method of treating or preventing an infection in a patient, comprising (i) administering to the patient the cells produced according to any of the methods described herein or (ii) administering to the patient any of the isolated populations of cells or pharmaceutical compositions described herein; in an amount effective to treat or prevent an infection in the patient.

It is further contemplated that the populations of T cells produced according to the inventive methods can be used in methods of treating or preventing an autoimmune condition in a patient. In this regard, an embodiment of the invention provides a method of treating or preventing an autoimmune condition in a patient, comprising (i) administering to the patient the cells produced according to any of the methods described herein or (ii) administering to the patient any of the isolated populations of cells or pharmaceutical compositions described herein; in an amount effective to treat or prevent an autoimmune condition in the patient.

One or more additional therapeutic agents can be co-administered to the patient. Use of “co-administering” herein means administering one or more additional therapeutic agents and the isolated population of cells sufficiently close in time such that the isolated population of cells can enhance the effect of one or more additional therapeutic agents, or vice versa. In this regard, the isolated population of cells can be administered first and the one or more additional therapeutic agents can be administered second, or vice versa. Alternatively, the isolated population of cells and the one or more additional therapeutic agents can be administered simultaneously. Additional therapeutic agents that may enhance the function of the isolated population of cells may include, for example, one or more cytokines or one or more antibodies (e.g., antibodies that inhibit PD-1 function). An exemplary therapeutic agent that can be co-administered with the isolated population of cells is IL-2. Without being bound to a particular theory or mechanism, it is believed that IL-2 may enhance the therapeutic effect of the isolated population of cells, e.g., T cells.

An embodiment of the invention further comprises lymphodepleting the patient prior to administering the isolated population of cells. Examples of lymphodepletion include, but may not be limited to, nonmyeloablative lymphodepleting chemotherapy, myeloablative lymphodepleting chemotherapy, total body irradiation, etc.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a patient. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset or recurrence of the disease, or a symptom or condition thereof.

The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70% or about 80%, about 90% or can be about 100%.

Unless stated otherwise, as used herein, the term “patient” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). It is preferred that the mammals are non-human primates, e.g., of the order Primates, Ceboids, or Simoids (monkeys) or of the order Anthropoids (humans and apes). In some embodiments, the mammal may be a mammal of the order Rodentia, such as mice and hamsters. In other embodiments, the mammal is not a mouse. Preferably, the mammal is a non-human primate or a human. An especially preferred mammal is the human.

With respect to the inventive methods, the cancer can be any cancer, including any of the cancers described herein with respect to other aspects of the invention.

### Examples of Non-Limiting Aspects of the Disclosure

Aspects, including embodiments, of the present subject matter described herein may be beneficial alone or in combination, with one or more other aspects or embodiments. Without limiting the foregoing description, certain non-limiting aspects of the disclosure numbered (1)-(20) are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A method of producing an isolated population of T cells for adoptive cell therapy, the method comprising:
a) providing a patient sample, wherein the patient sample is (1) a sample containing T cells from a patient having a tumor, (2) a sample containing T cells from a patient with an infection, or (3) a sample containing T cells from a patient with an autoimmune condition, wherein the T cells comprise at least one T Cell Receptor (TCR), wherein the TCR comprises an alpha-chain and beta-chain pair;
b) separating T cells from other cells of the patient sample of a) to produce separated T cells;
c) culturing the separated T cells of b) to produce T cell induced pluripotent stem cells (iPSCs);
d) culturing the T cell iPSCs of c) to produce CD4⁻CD8⁻(double negative) T cells;
e) screening the CD4⁻CD8⁻(double negative) T cells to identify one or more CD4⁻CD8⁻(double negative) T cells that comprise a TCR alpha-chain and beta-chain pair having antigenic specificity for an antigen of interest, wherein the antigen of interest is (1) a cancer antigen, (2) a pathogen antigen, or (3) an antigen causing autoimmunity; and
f) differentiating the double negative T cell(s) identified in e) as comprising the TCR alpha-chain and beta-chain pair having antigenic specificity for the antigen of interest into naïve T cells and expanding the number of naïve T cells to produce an isolated population of T cells for adoptive cell therapy.

(2) The method according to aspect (1), further comprising obtaining sequence(s) which encode the alpha-chain and beta-chain pair of the TCR.

(3) The method according to aspect (2), further comprising transducing naïve peripheral blood mononuclear cells (PMBC) with the sequence of the alpha-chain and beta-chain pair of the TCR to provide an isolated population of cells for adoptive cell therapy.

(4) The method according to any one of aspects (1)-(3), wherein the T cells of a) are tumor infiltrating lymphocytes (TIL).

(5) The method according to any one of aspects (1)-(4), further comprising culturing the T cells of c) in the presence of interleukin-2 (IL-2), interleukin-7 (IL-7), interleukin-15 (IL-15), interleukin-12 (IL-12), or a combination of two or more of the foregoing.

(6) The method according to any one of claims 1)-(5), wherein the antigen of interest is a cancer antigen.

(7) The method according to any one of claims 1)-(6), wherein the TCR alpha-chain and beta-chain pair has specificity for a cancer antigen.

(8) The method according to any one of aspects (3)-(7), wherein the naïve PMBC are transduced with the sequence of the alpha-chain and beta-chain pair of the TCR using one or more vectors.

(9) The method according to aspect (8), wherein the vector(s) is/are viral vector(s).

(10) The method according to aspect (8) or (9), wherein the vector(s) is/are retroviral vector(s), gamma-retroviral vector(s), or lentiviral vector(s).

(11) The method according to any one of aspects (3)-(10), wherein the PMBCs are peripheral blood lymphocytes (PBLs), B cells, dendritic cells, or a combination of two or more of the foregoing.

(12) An isolated population of T cells produced by the method according to any one of aspects (1)-(11).

(13) A pharmaceutical composition comprising the isolated population of T cells of aspect (12) and a pharmaceutically acceptable carrier.

(14) A method of treating or preventing cancer in a patient, the method comprising producing an isolated T cell population according to the method of any one of aspects (1)-(11), and administering the isolated T cell population to the patient in an amount effective to treat or prevent cancer in the patient.

(15) A method of treating or preventing an infection in a patient, the method comprising producing an isolated T cell population according to the method of any one of aspects (1)-(11), and administering the isolated T cell population to the patient in an amount effective to treat or prevent the infection in the patient.

(16) A method of treating or preventing an autoimmunity condition in a patient, the method comprising producing an isolated T cell population according to the method of any one of aspects (1)-(11), and administering the isolated T cell population to the patient in an amount effective to treat or prevent an autoimmunity condition in the patient.

(17) The T cell population isolated according to the method of any one of aspects (1)-(11), or the composition of aspect (13), for use in the treatment or prevention of cancer in a patient.

(18) The T cell population isolated according to the method of any one of aspects (1)-(11), or the composition of aspect (13), for use in the treatment or prevention of an infection in a patient.

(19) The T cell population isolated according to the method of any one of aspects (1)-(11), or the composition of aspect (13), for use in the treatment or prevention of an autoimmunity condition in a patient.

### EXAMPLES

The following examples further illustrate the invention but, of course, should not be construed as in any way limiting its scope.

### Example 1

This example demonstrates that an isolated population of T cells for ACT can be successfully produced using iPSCs.

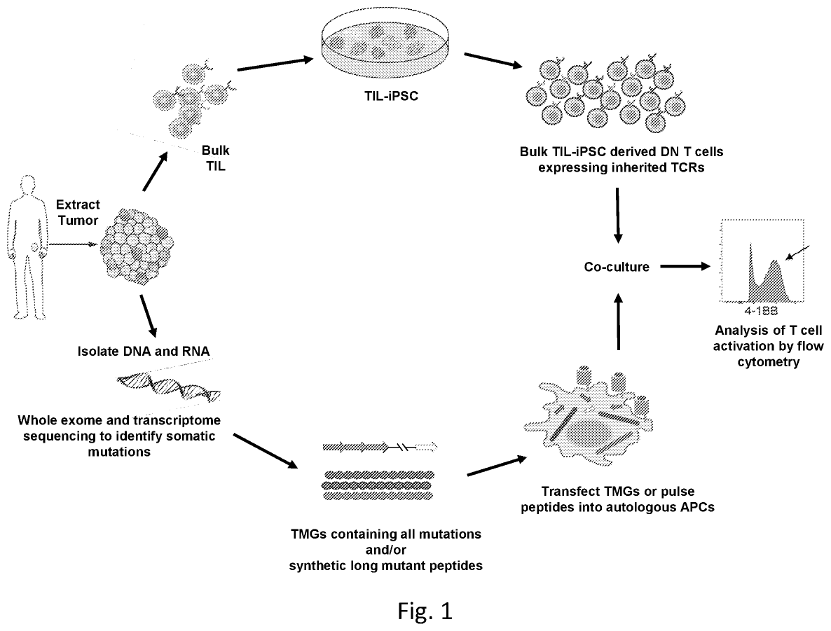

First, a tumor sample was provided from a patient that contained T cells having TCRs with an alpha-chain and beta-chain pair. The T cells were then separated from the tumor cells using FACS. The T cells were then cultured using a standard technique to produce T cell iPSCs. The T cell iPSCs were then cultured using a standard technique to produce CD4⁻CD8⁻(double negative) T cells. The CD4⁻CD8⁻ double negative T cells were then screened to identify the T cells that had the TCRs with an alpha-chain and beta-chain pair of interest (the ones having antigenic specificity for the patient's tumor cells). The T cells of interest were then differentiated into naïve T cells and were expanded to produce a large population of naïve T cells. The method is illustrated in FIG. 1.

### Example 2

This example demonstrates that somatic cells can be reprogrammed into an embryonic stem cell-like stage, the numbers of which can be indefinitely expanded while retaining the potential to differentiate into any type of somatic cell, using iPSC technology.

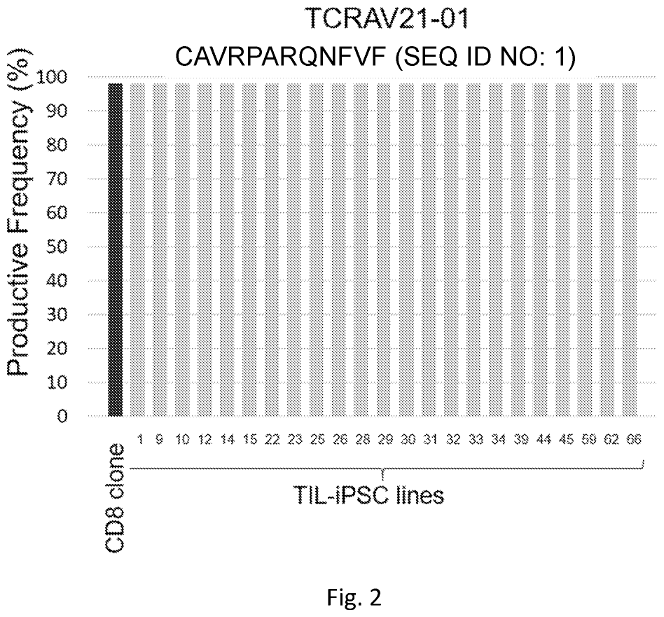

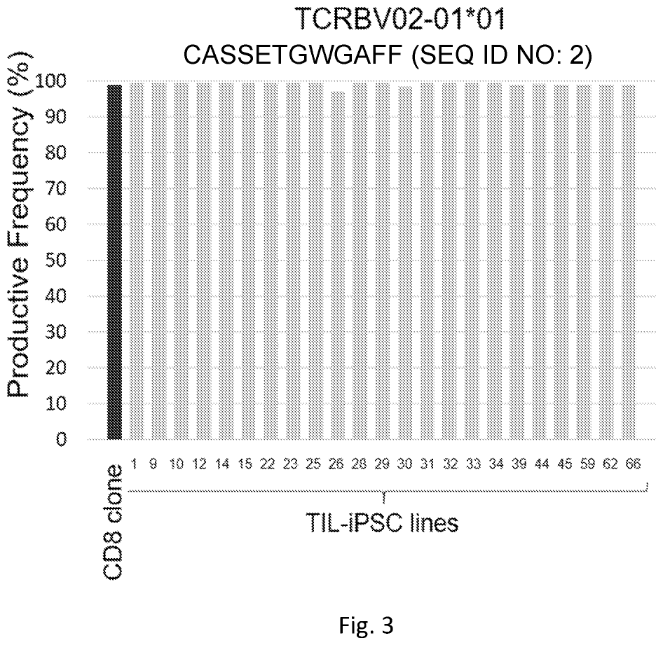

Twenty-three iPSC lines were produced from TIL that were specific for mutated GBAS peptide. All of the TIL-iPSC lines generated showed only one inherited arrangement of a TCR alpha-chain and beta-chain pair, which was identical to that of the cell source. FIG. 2 shows the percentage of productive frequency for CD8 alone or with TIL-iPSC lines for TCR AV21-01. FIG. 3 shows the percentage of productive frequency for CD8 alone or TIL-iPSC lines for TCR BV02-01*01.

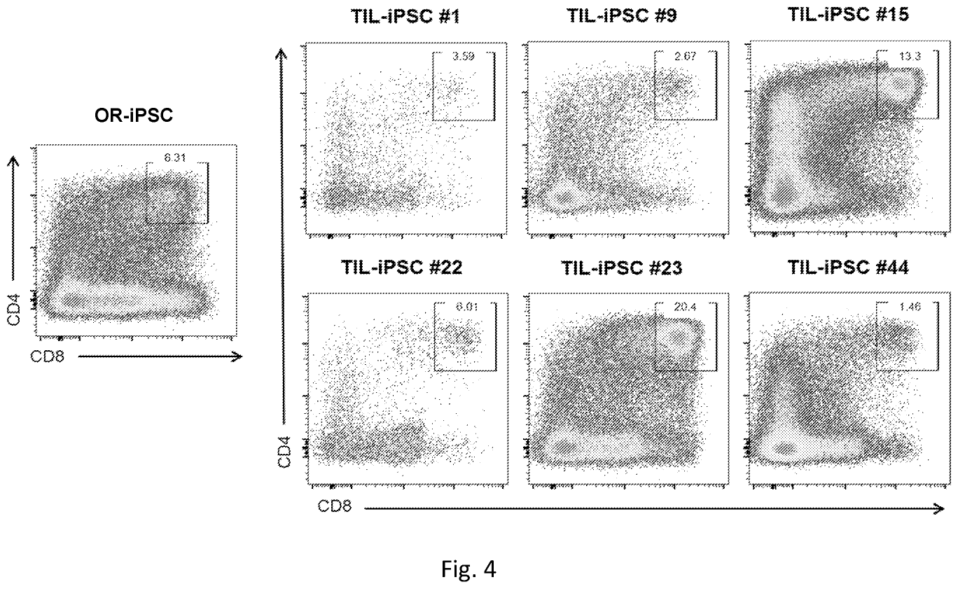

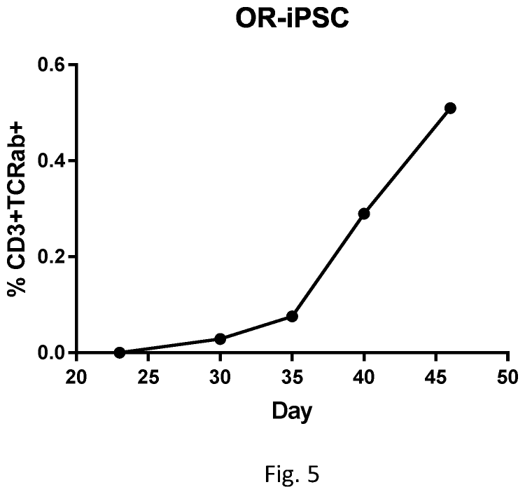

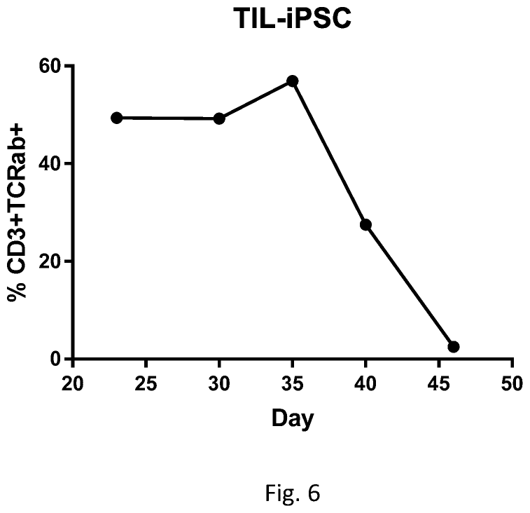

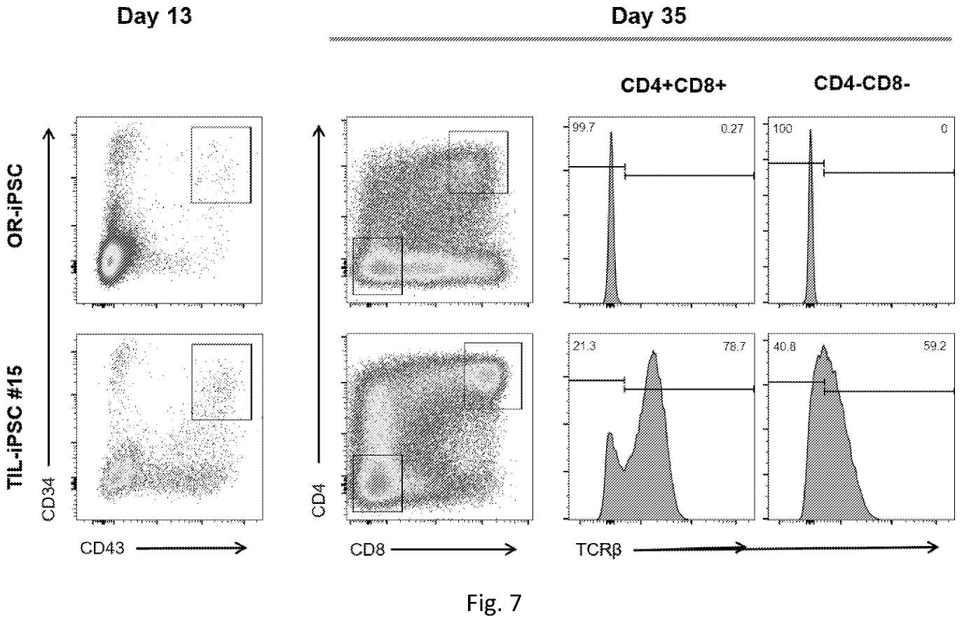

Five of the 23 iPSC lines were differentiated into T lineage cells in vitro. FIG. 4 shows FACS data illustrating the differentiation. All 5 of the selected TIL-iPSC lines produced CD4+CD8+ double positive T cells by day 35. FIG. 5 shows the percentage of CD3+TCRab+open-repertoire iPSC (OR-iPSC) per day. FIG. 6 shows the percentage of CD3+TCRab+TIL-iPSC per day. FIG. 7 shows FACS data illustrating the precocious expression of TCR complex in human TIL-iPSC derived immature T cells (TIL-iPSC line #15).

### Example 3

This example demonstrates that TIL-iPSC derived CD4⁺CD8⁺(double positive) T cells do not show specificity against tumor mutations but that TIL-iPSC derived CD4⁻CD8⁻(double negative) T cells have high tumor-antigen specific reactivity.

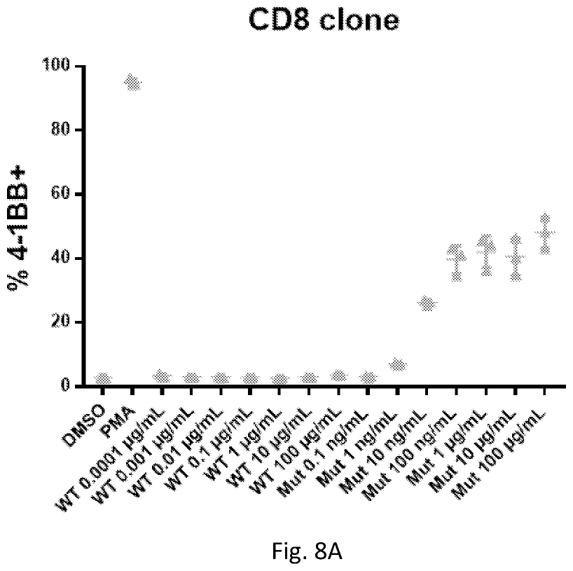

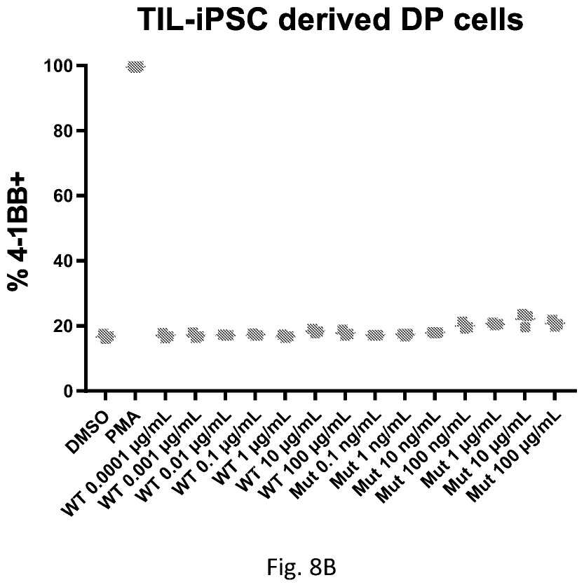

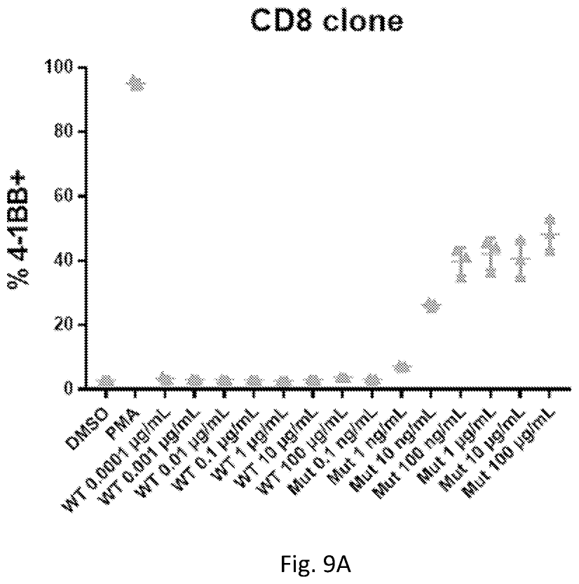

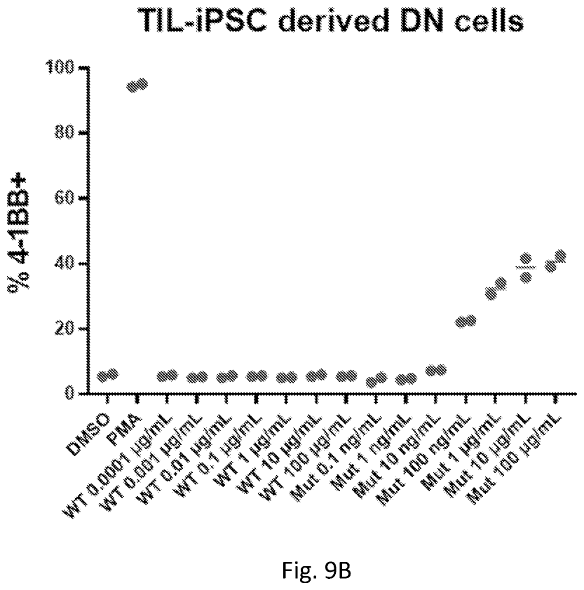

The double positive T cells were activated (4-1BB+) by PMA stimulation but not by mutated peptide as compared to the TIL cell source, as seen in FIG. 8A (CD8 alone) and 8B (TIL-iPSC derived double positive cells). The double negative T cells were activated (4-1BB+) by PMA stimulation and responded to mutated peptide but not to wild type peptide as the parental TILs did, as seen in FIG. 9A (CD8 alone) and 9B (TIL-iPSC derived double negative cells).

### Example 4

This example demonstrates that CD4+CD8+(double positive) T cells cannot be used for screening by IFNγ production, but double negative cells can be used for such screening.

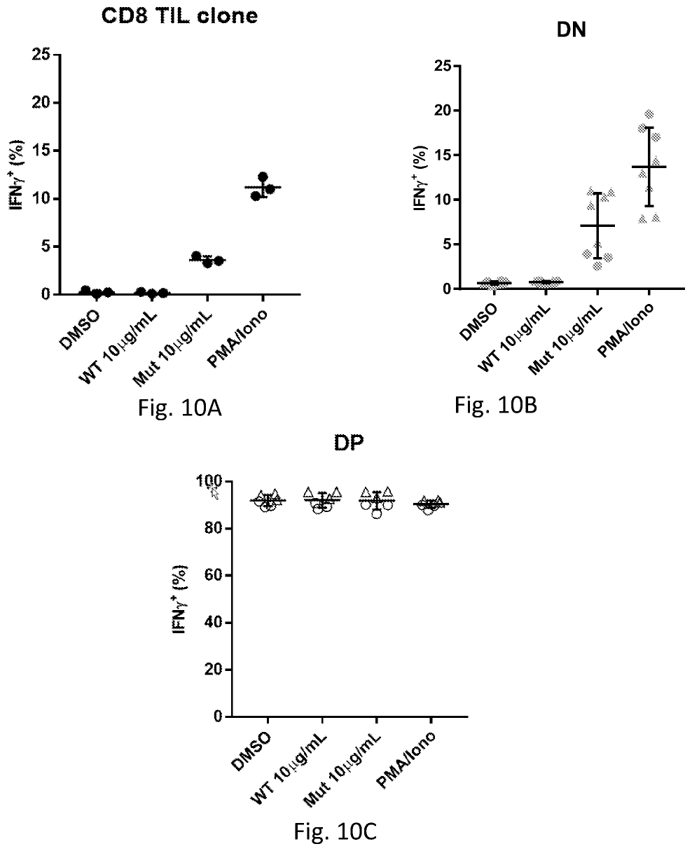

Day 37 TIL-iPSC derived T cells were sorted according to CD4-CD8− (double negative, DN) and CD4+CD8+(double positive, DP) markers. These populations, as well as the original CD8 TIL clone, were co-cultured with allogenic B cells pulsed with mutant peptide, wild type peptide or DMSO (negative control). Positive control was PMA/ionomycin. After a 6 hour co-culture, cells were washed three times with PBS and stained for intracellular cytokine production. Double negative cells showed IFNγ production in response to mutant peptide but not wild type peptide (FIG. 10B), similar to CD8 TIL clone (FIG. 10A). DP cells showed nonspecific production of INFγ across all conditions (FIG. 10C). Flow cytometric analysis was gated on live lymphocytes, CD3+. N=1 for CD8 TIL clone. N=2 for DN and DP. For all experiments, n was 3.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing an isolated population of T cells for adoptive cell therapy, the method comprising:
a) providing a patient sample, wherein the patient sample is (1) a sample containing T cells from a patient having a tumor, (2) a sample containing T cells from a patient with an infection, or (3) a sample containing T cells from a patient with an autoimmune condition, wherein the T cells comprise at least one T Cell Receptor (TCR), wherein the TCR comprises an alpha-chain and beta-chain pair;
b) separating T cells from other cells of the patient sample of a) to produce separated T cells;
c) culturing the separated T cells of b) to produce T cell induced pluripotent stem cells (iPSCs);
d) culturing the T cell iPSCs of c) to produce CD4⁻CD8⁻(double negative) T cells;
e) screening the CD4⁻CD8⁻(double negative) T cells to identify one or more CD4⁻CD8⁻(double negative) T cells that comprise a TCR alpha-chain and beta-chain pair having antigenic specificity for an antigen of interest, wherein the antigen of interest is (1) a cancer antigen, (2) a pathogen antigen, or (3) an antigen causing autoimmunity; and
f) differentiating the double negative T cell(s) identified in e) as comprising the TCR alpha-chain and beta-chain pair having antigenic specificity for the antigen of interest into naïve T cells and expanding the number of naïve T cells to produce an isolated population of T cells for adoptive cell therapy.

2. The method according to claim 1, further comprising obtaining sequence(s) which encode the alpha-chain and beta-chain pair of the TCR.

3. The method according to claim 2, further comprising transducing naïve peripheral blood mononuclear cells (PMBC) (PBMC) with the sequence of the alpha-chain and beta-chain pair of the TCR to provide an isolated population of cells for adoptive cell therapy.

4. The method according to claim 1, wherein the T cells of a) are tumor infiltrating lymphocytes (TIL).

5. The method according to claim 1, further comprising culturing the T cells of c) in the presence of interleukin-2 (IL-2), interleukin-7 (IL-7), interleukin-15 (IL-15), interleukin-12 (IL-12), or a combination of two or more of the foregoing.

6. The method according to claim 1, wherein the antigen of interest is a cancer antigen.

7. The method according to claim 1, wherein the TCR alpha-chain and beta-chain pair has specificity for a cancer antigen.

8. The method according to claim 3, wherein the naïve PBMC are transduced with the sequence of the alpha-chain and beta-chain pair of the TCR using one or more vectors.

9. The method according to claim 8, wherein the vector(s) is/are viral vector(s).

10. The method according to claim 8, wherein the vector(s) is/are retroviral vector(s), gamma-retroviral vector(s), or lentiviral vector(s).

11. The method according to claim 3, wherein the PBMC are peripheral blood lymphocytes (PBLs), B cells, dendritic cells, or a combination of two or more of the foregoing.

12. An isolated population of T cells produced by the method according to claim 1.

13. A pharmaceutical composition comprising the isolated population of T cells of claim 12 and a pharmaceutically acceptable carrier.

14-16. (canceled)

17. A method of treating or preventing cancer in a patient, the method comprising producing an isolated T cell population according to the method of claim 1 and administering the isolated T cell population to the patient in an amount effective to treat or prevent cancer in the patient.

18. A method of treating or preventing an infection in a patient, the method comprising producing an isolated T cell population according to the method of claim 1 and administering the isolated T cell population to the patient in an amount effective to treat or prevent the infection in the patient.

19. A method of treating or preventing an autoimmunity condition in a patient, the method comprising producing an isolated T cell population according to the method of claim 1 and administering the isolated T cell population to the patient in an amount effective to treat or prevent an autoimmunity condition in the patient.
